(19) United States
(12) Patent Application Publication
(10) Pub. No.: US 2023/0392153 A1
(43) Pub. Date: Dec. 7, 2023

(54) SUMO Targeting Morpholinos as both Enhancers of Anti-Cancer Effects of Chemotherapeutic Treatments and Preventative Agents Against the Establishment of Chemoresistance

(71) Applicant: Board of Regents, The University of Texas System, Austin, TX (US)

(72) Inventor: German Rosas-Acosta, El Paso, TX (US)

(21) Appl. No.: 18/330,563

(22) Filed: Jun. 7, 2023

Related U.S. Application Data
(60) Provisional application No. 63/365,965, filed on Jun. 7, 2022.

(51) Int. Cl. C12N 15/113

(52) U.S. Cl. CPC C12N 15/113; C12N 2310/3233; C12N 2310/11

(57) ABSTRACT

A method of preventing, treating or delaying progression of cancer, includes administering a therapeutically effective amount of a compound comprising at least 2 morpholinos to a patient in need thereof. These can be vivo-morpholinos that include an octa-guanidine dendrimer. The at least 2 vivo-morpholinos can include an S2E3Acc comprising the sequence of SEQ ID NO: 1 or 3 or 5; and an S2E3Don comprising the sequence of SEQ ID NO: 2 or 4 or 6.

### CROSS-REFERENCE TO RELATED APPLICATION

Referring to the application data sheet filed herewith, this application claims a benefit of priority under 35 U.S.C. 119(e) from co-pending provisional patent application U.S. Ser. No. 63/365,965, filed Jun. 7, 2022, the entire contents of which are hereby expressly incorporated herein by reference for all purposes.

### STATEMENT AS TO RIGHTS TO INVENTIONS MADE UNDER FEDERALLY-SPONSORED RESEARCH OR DEVELOPMENT

This invention was made with government support under #W81XWH2010088 awarded by Department of Defense US Army Medical Research Program. The government has certain rights in the invention.

### REFERENCE TO AN ELECTRONIC SEQUENCE LISTING

The contents of the electronic sequence listing (UTEP2022-014_take2finalsequences.xml; Size: 9142 bytes; and Date of Creation: Jun. 1, 2023) is herein incorporated by reference in its entirety for all purposes.

### REFERENCE TO APPENDIX

An appendix is included in this application by way of attachment, the totality of which is hereby incorporated by reference for all purposes as an integral part of this application. The appendix includes an English-language sequence listing as backup. are specified as media type pdf of hardcopy printout of WIPO validated sequence.

### BACKGROUND

The SUMOylation system has been recognized as a very promising target for the development of novel therapies against cancer. Numerous tumors are known to exhibit increases in specific components of the SUMOylation system and inactivating such components by knock-out, knock-down, or chemical inhibitor strategies has demonstrated the relevant role played by those components in ensuring tumor survival. Additionally, an over-active SUMOylation system has been demonstrated to play a critical role in the establishment of resistance against chemotherapeutic agents. In view of these facts, chemical inhibitors capable of blocking cellular SUMOylation were postulated to be viable anti-cancer therapeutic agents. The large body of data supporting this hypothesis provided the empirical support that has resulted in the use of such SUMO inhibitors in human trials. While the data provided by such trials are still being collected, it is likely that the use of inhibitors of the enzymatic players of the SUMOylation system may produce side effects similar to those associated with other chemotherapeutic agents.

Synthetic antisense oligonucleotides, and specifically those readily up-taken by mammalian cells, such as vivo-morpholinos and similarly modified morpholinos, are among the most favorable molecules for human therapies. Morpholinos contain standard DNA bases that form canonical Watson-Crick base pairs with their target primary transcripts, and a morpholino phosphorodiamidate backbone that exhibits no charge and displays minimal protein binding capacity, rendering them resistant to all DNases and RNases present in cellular and extracellular environments. These characteristics provide morpholinos with exquisite target specificity and unmatched biological stability. The outstanding qualities of morpholinos as tools for human therapies are demonstrated by the relatively recent approval by the FDA of four different morpholino antisense oligomers, namely eteplirsen, golodirsen, viltolarsen, and casimersen, for the treatment of Duchenne Muscular Dystrophy (DMD) in the United States.

Heretofore, the requirement(s) referred to above has(have) not been fully met. In view of the foregoing, there is a need in the art for a solution that (simultaneously) solves this (both of these)(all of these) problem(s).

### SUMMARY

There is a need for the following embodiments of the present disclosure. Of course, the present disclosure is not limited to these embodiments.

Considering all the information provided above, the six-morpholinos cocktail herein presented, which is composed of three pairs of gene-specific morpholinos, namely S1E2Acc and S1E2Don (targeting SUMO1), S2E3Acc and S2E3Don (targeting SUMO2), and S3E3Acc and S3E3Don (targeting SUMO3), could be rapidly moved from animal models to clinical trials. The 6-morpholino cocktail, hereafter referred to as the S123ESM (which stands for SUMO1, 2, & 3 Exon Skipping Morpholinos), is likely to exhibit prolonged biological activity and exquisite specificity, targeting exclusively the primary transcripts for SUMO1, SUMO2, and SUMO3, and forcing exon-skipping alternative splicing events that drive the production of non-conjugatable SUMO proteins, namely SUMO1α, SUMO2α, SUMO3b.

The data supporting the development of these morpholinos was obtained during studies performed by the inventor's research group (Dr. Rosas-Acosta's laboratory). This data has not been published nor released in any form. The specific design of these exon-skipping morpholinos requires intimate knowledge of the molecular regulation of the SUMO genes, which is not readily available. Furthermore, the application of the morpholino exon-skipping technology to the down-regulation of the SUMO system or any other Ubiquitin-like post-translational modification system is unprecedented. This demonstrates that it is not an obvious application of an easily accessible approach.

According to an embodiment of the present disclosure, a method of preventing, treating, or delaying the progression of cancer, comprises administering a therapeutically effective amount of the 6 synthetic antisense oligonucleotides included in the S123ESM mix as indicated above, or any mix of the 3 pairs of antisense oligonucleotides that form the S123ESM mix, to a patient in need thereof. The preferred chemical form proposed is that of vivo-morpholinos, which includes the morpholino chemistry and an octa-guanidine dendrimer at the 3′-end, but includes any other chemical structures that target the same sequences via synthetic antisense oligonucleotides. The sequences included are:

SUMO1: S1E2Acc comprising the sequence of SEQ ID NO:1; and S1E2Don comprising the sequence of SEQ ID NO:2.

SUMO2: S2E3Acc comprising the sequence of SEQ ID NO: 3; and S2E3Don comprising the sequence of SEQ ID NO: 4.

SUMO3: S3E3Acc comprising the sequence of SEQ ID NO: 5; and S3E3Don comprising the sequence of SEQ ID NO: 6.

According to another embodiment of the present disclosure, a composition of matter comprises: a compound comprising at least 2 morpholinos targeting a single SUMO-associated gene. These can be vivo-morpholinos that include an octa-guanidine dendrimer. The at least 2 vivo-morpholinos can include S1E2Acc comprising the sequence of SEQ ID NO:1; and an S1E2Don comprising the sequence of SEQ ID NO:2; OR, S2E3Acc comprising the sequence of SEQ ID NO: 3; and S2E3Don comprising the sequence of SEQ ID NO: 4; OR, S3E3Acc comprising the sequence of SEQ ID NO: 5; and S3E3Don comprising the sequence of SEQ ID NO: 6.

According to another embodiment of the present disclosure, a method comprises quantifying the specific SUMO-related transcript comprising: maintaining mammalian cells in the presence of at least 2 vivo-morpholinos; and performing qRT-PCR reactions on RNA isolated from mammalian cells to assess copy number estimates per unit mass of total RNA for at least one mature mRNA transcript variant derived from the SUMO gene selected. To that end, a set of specific primers for transcript-specific amplification is required from the group consisting of S2V1 and/or S2V2. The at least 2 vivo-morpholinos can comprise: an S2E3Acc comprising the sequence of SEQ ID NO: 3; and an S2E3Don comprising the sequence of SEQ ID NO: 4. The method can include providing, before performing, a forward primer S2V1V2.FW comprising the sequence of SEQ ID NO: 7; providing, before performing, a reverse primer S2V1.RV comprising the sequence of SEQ ID NO: 8; providing, before performing, a forward primer S2V1V2.FW comprising the sequence of SEQ ID NO: 7; and providing, before performing, a reverse primer S2V2.RV comprising the sequence of SEQ ID NO: 9.

These, and other, embodiments of the present disclosure will be better appreciated and understood when considered in conjunction with the following description and the accompanying drawings. It should be understood, however, that the following description, while indicating various embodiments of the present disclosure and numerous specific details thereof, is given for the purpose of illustration and does not imply limitation. Many substitutions, modifications, additions and/or rearrangements may be made within the scope of embodiments of the present disclosure, and embodiments of the present disclosure include all such substitutions, modifications, additions and/or rearrangements.

### BRIEF DESCRIPTION OF THE DRAWINGS

The drawings accompanying and forming part of this specification are included to depict certain embodiments of the present disclosure. A clearer concept of the embodiments described in this application will be readily apparent by referring to the exemplary, and therefore nonlimiting, embodiments illustrated in the drawings (wherein identical reference numerals (if they occur in more than one view) designate the same elements).

The described embodiments may be better understood by reference to one or more of these drawings in combination with the following description presented herein. It should be noted that the features illustrated in the drawings are not necessarily drawn to scale.

FIG. 1 is a representation of the SUMO1 gene and its exons, and the specific sequences targeted by S1E2Acc and S1E2Don.

FIG. 2 is a representation of the SUMO2 gene and its exons, and the specific sequences targeted by S2E3Acc and S2E3Don.

FIG. 3 is a representation of the SUMO3 gene and its exons, and the specific sequences targeted by S3E3Acc and S3E3Don.

FIG. 4 is a representation of the basic chemical structure of an unmodified morpholino and a vivo-morpholino.

FIG. 5 is a representation of a quantitative analysis of the SUMO2 transcript variants present in A549 cells under normal conditions as well as upon a 24 hour treatment with the vivo-morpholinos S2E3Acc and S2E3Don.

### DETAILED DESCRIPTION

Embodiments presented in the present disclosure and the various features and advantageous details thereof are explained more fully with reference to the nonlimiting embodiments that are illustrated in the accompanying drawings and detailed in the following description. Descriptions of well-known materials, techniques, components, and equipment are omitted so as not to unnecessarily obscure the embodiments of the present disclosure in detail. It should be understood, however, that the detailed description and the specific examples are given by way of illustration only and not by way of limitation. Various substitutions, modifications, additions, and/or rearrangements within the scope of the underlying inventive concept will become apparent to those skilled in the art from this disclosure.

The below-referenced U.S. Patent(s) and/or U.S. Patent Application(s) disclose embodiments that are useful for the purposes for which they are intended. The entire contents of U.S. Pat. Application No. 2012/0246757, published 2012 Sep. 27, are hereby expressly incorporated by reference herein for all purposes.

Embodiments of this disclosure describe the specific sequence of six morpholinos that, when given together to human cells, alter the proportion of mature transcripts produced by the main SUMO genes expressed in humans. In human cells, there are five different SUMO genes, SUMO1-5, which code for five different proteins, SUMO1-5. SUMO4 and SUMO5 are expressed at very low levels and in a limited set of tissues and therefore are considered of secondary relevance. The SUMO1 gene produces three main mature mRNAs: SUMO1 variant 1 (S1V1), SUMO1 variant 2 (S1V2), and SUMO1 variant 3 (S1V3). The first two variants (S1V1 and S1V2) code for the prototypical SUMO1 protein; S1V3 codes for a non-conjugatable form of SUMO1 known as SUMO1 alpha (SUMO1α). The SUMO2 gene produces two main mature mRNAs: i) SUMO2 variant 1 (S2V1), which codes for the prototypical SUMO2 protein; and ii) SUMO2 variant 2 (S2V2), which codes for a non-conjugatable form of SUMO2, hereafter referred to as SUMO2 alpha (SUMO2α). The SUMO3 gene produces two main mature mRNAs: i) SUMO3 variant 1 (S3V1), which codes for the prototypical SUMO3 protein; and ii) SUMO3 variant 2 (S3V2), which codes for SUMO3 alpha (SUMO3a), an isoform of SUMO3 containing 38 additional amino acid residues. Under normal conditions, the S2V1 mRNA is produced at a CNE_{100 ng} (Copy Number Estimate per 100 ng of total RNA) of ˜2 million copies, whereas the S2V2 mRNA is produced at a CNE_{100 ng} of ˜15 thousand copies, thus accounting for an approximate difference of about 100 fold, with S2V1 being vastly more abundant in the cell than S2V2. Similarly, the S1V1 and S1V2 transcripts are vastly more abundant than S1V3, and S3V1 is also substantially more abundant than S3V2.

Treatment with the different morpholino pairs herein described drastically alters the proportion of the SUMO transcripts, increasing that of the transcripts coding for the SUMO alphas. Specifically, the morpholinos targeting SUMO2, S2E3Acc and S2E3Don, alter the proportion of S2V1:S2V2 from its original proportion of 2 million:15 thousand to a new proportion of about 45 thousand:2.5 million. The effect of this switch in the proportion of S2V1:S2V2 is that SUMO2α becomes by far the predominant SUMO in the cell. As SUMO2α is not functional as a modifier, i.e., it cannot be conjugated to any target, this leads to a dramatic decrease in overall SUMO conjugation in the cell, thus negatively affecting all cellular activities that are regulated by SUMOylation, including DNA repair, progression through mitosis, DNA replication, and regulation of cytoskeletal structures. Cell survival and cell multiplication are therefore predicted to be substantially compromised.

Observations related to the effects mediated by the morpholinos herein described:

There are three main SUMO genes in human cells, SUMO1, SUMO2, and SUMO3. These genes code for three different paralog proteins, SUMO1, SUMO2, and SUMO3, which provide specific functions that appear only partially overlapping. The predominant type of SUMO expressed in any specific organ appears to be organ-specific, so SUMO1 is the main SUMO expressed in some organs, whereas SUMO2 and SUMO3 are the predominant SUMOs in other organs. However, in all tumor-derived cell lines analyzed to date, the predominant SUMO gene expressed is SUMO2. Thus, there appears to be some correlation between SUMO2 expression and tumor development, although it is not clear whether increased SUMO2 expression promotes tumor development or whether tumor development promotes SUMO2 expression. Either way, in all tumor-derived cell lines currently available, the predominant transcript found is the one coding for SUMO2 and it is about 100-fold more abundant than the transcripts for either SUMO1 or SUMO3.

Considering that there is ample proof that the SUMOylation system is overactive in most tumors, that shutting-down the SUMOylation system is known to exert anti-tumor effects, that there are two ongoing clinical trials assessing the use of SUMO system inhibitors for the treatment of cancer, and that an active SUMOylation system appears essential for the establishment of resistance to chemotherapy, the six morpholinos herein presented will exert two main activities: i) inactivate the SUMOylation system, therefore increasing tumor cell death upon chemotherapeutic treatment; and, ii) minimize the odds that tumor cells may develop resistance to the chemotherapeutic agent during chemotherapy.

Details Related to the Morpholinos Disclosed in this Application:

The morpholinos disclosed in this patent application are named S1E2Acc, S1E2Don, S2E3Acc, S2E3Don, S3E3Acc, and S3E3Don. Their names indicate that they specifically target a given SUMO transcript and exon at either the splicing acceptor site (Acc), or the splicing donor site (Don). For example, S1E2Acc targets SUMO1 exon 2 at the splicing acceptor site.

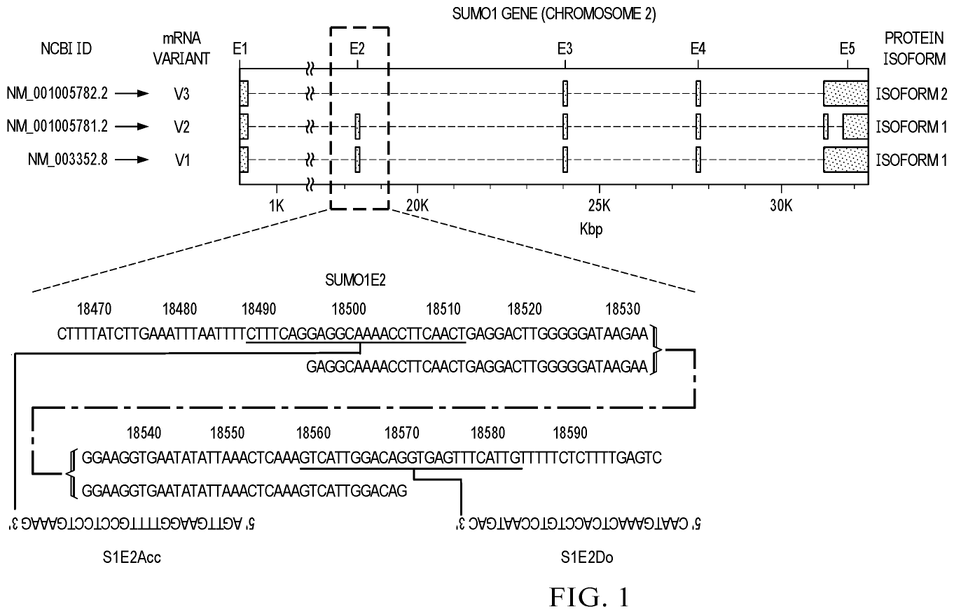

The specific sequences targeted by these vivo-morpholinos are represented in FIGS. 1-3.

Referring to FIG. 1, a representation of the location of Exon 2 within the SUMO1 gene sequence and the specific sequences targeted by S1E2Acc and S1E2Don is presented. The sequence 5′ AGTTGAAGGTTTTGCCTCCTGAAAG 3′ is included in S1E2Acc. The sequence CAATGAAACTCACCTGTCCAATGAC 3′ is included in S1E2Don.

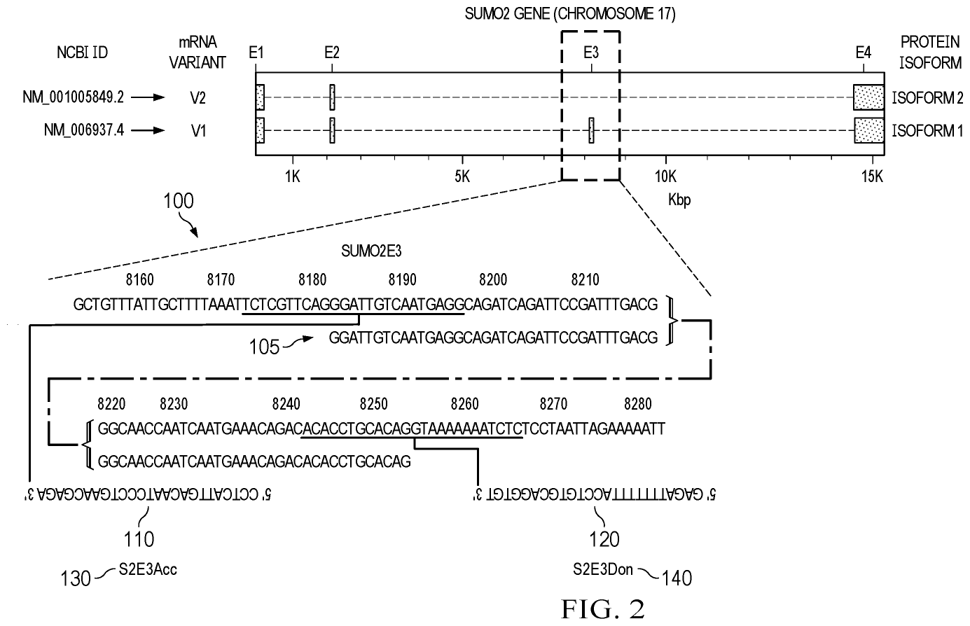

Referring to FIG. 2, a representation of the location of Exon 3 105 within the SUMO2 gene sequence 100 and the specific sequences targeted by S2E3Acc 130 and S2E3Don 140 is presented. The sequence 5′ CCTCATTGACAATCCCTGAACGAGA 3′ 110 is included in S2E3Acc 130. The sequence 5′ GAGATTTTTTTACCTGTGCAGGTGT 3′ 120 is included in S2E3Don 140.

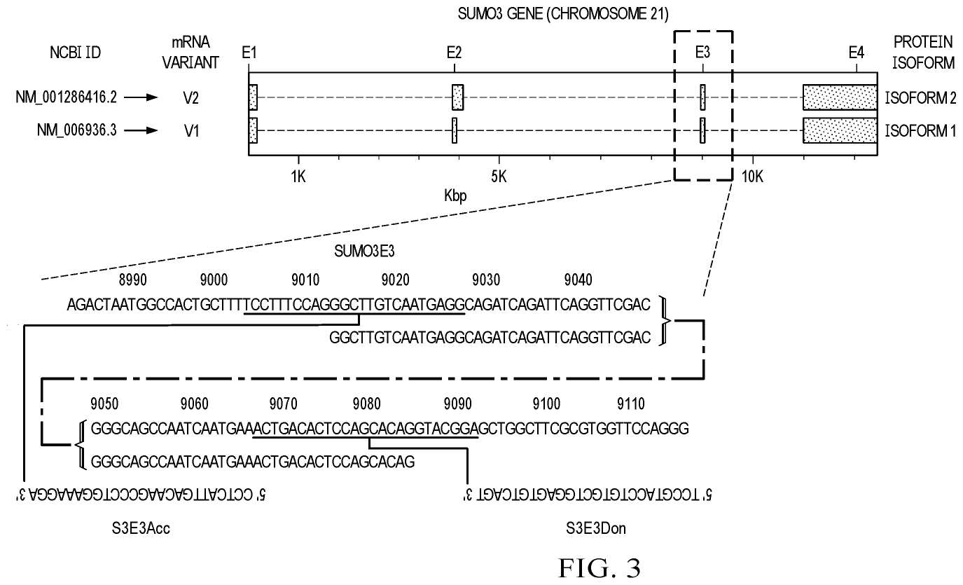

Referring to FIG. 3, a representation of the location of Exon 3 within the SUMO3 gene sequence and the specific sequences targeted by S3E3Acc and S3E3Don is presented. The sequence 5′ CCTCATTGACAAGCCCTGGAAAGGA 3′ is included in S3E3Acc. The sequence TCCGTACCTGTGCTGGAGTGTCAGT 3′ 120 is included in S3E3Don

The 4-digit numbers in FIG. 1-3 indicate base pairs from the transcriptional start site for the respective SUMO gene. The nucleotide sequence shown right under the numbers corresponds to the sequence of the respective SUMO gene, and the nucleotide sequence shown below indicates the sequence of the exon targeted by the morpholinos. The sequences of the morpholinos are shown underneath the Exon sequences; since the morpholinos are complementary to the transcript, they are shown upside down, in their 5′ to 3′ direction. Notice that the morpholinos target the boundary between the introns and exons, with S1E2Acc, S2E3Acc, and S3E3Acc being complementary to the intron-exon sequence at the splicing acceptor region, and S1E2Don, S2E3Don, and S3E3Don being complementary to the exon-intron sequence at the splicing donor region.

5.2. Exemplary Sequences:

Vivo-morpholino S1E2Acc:
SEQ ID NO: 1
5′ AGTTGAAGGTTTTGCCTCCTGAAAG 3′
Vivo-morpholino S1E2Don:
SEQ ID NO: 2
5′ CAATGAAACTCACCTGTCCAATGAC 3′.
Vivo-morpholino S2E3Acc:
SEQ ID NO: 3
5′ CCTCATTGACAATCCCTGAACGAGA 3′
Vivo-morpholino S2E3Don:
SEQ ID NO: 4
5′ GAGATTTTTTTACCTGTGCAGGTGT 3′
Vivo-morpholino S3E3Acc:
SEQ ID NO: 5
5′ CCTCATTGACAAGCCCTGGAAAGGA 3′
Vivo-morpholino S3E3Don:
SEQ ID NO: 6
5′ TCCGTACCTGTGCTGGAGTGTCAGT 3′

5.3. Chemical structures:

This disclosure is intended to cover all morpholino-related structures containing the DNA base sequences presented above. Therefore, it includes morpholinos modified by the addition of extra chemical groups to increase their cellular uptake and/or penetration, as well as morpholinos containing no additional modification, or no modification at all, and other synthetic oligonucleotides targeting the same sequences with the purpose of affecting the splicing process undergone by the primary transcripts of the SUMO1-3 genes.

The type of morpholinos used in the experiments presented in this disclosure were the ones referred as vivo-morpholinos. Vivo-morpholinos differ from normal unmodified morpholinos in that they contain a octa-guanidine dendrimer attached to the region corresponding to the 3′ end of the molecule. The octa-guanidine dendrimer is chemically equivalent to an arginine rich peptide and therefore confers to the vivo-morpholino molecule the cell permeability characteristic of cell-permeable peptides. This results in substantial increases in the cellular uptake of the vivo-morpholino as compared to that of an unmodified morpholino. The basic structures of unmodified morpholinos and vivo-morpholinos are shown in FIG. 2.

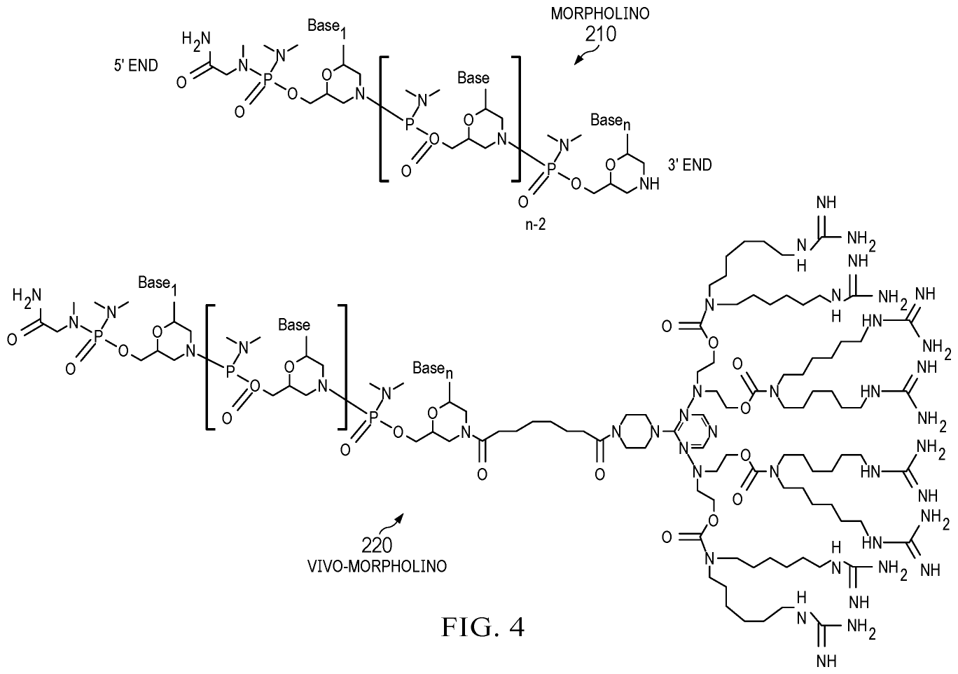

Referring to FIG. 4, basic chemical structures of an unmodified morpholino 210 and a vivo-morpholino 220 are presented. The 5′ end of the morpholinos presented is at the left, as it is conventionally used for all nucleic acids. Notice that the even though the backbone of the morpholinos is substantially different from that of DNA and RNA molecules, the spacing between the bases is exactly the same as it is in DNA and RNA. Also, notice the octa-guanidine dendrimer located at the 3′ end of the Vivo-morpholino.

Data Demonstrating the Effect Mediated by Treatment with S2E3Acc & S2E3Don:

Protocols Followed:

Made to order vivo-morpholinos are readily available and can be procured from Gene Tools, LLC (Philomath, OR) based on specified embodiments of this disclosure, and reconstituted at a final concentration of 400 μM using RNase & DNase-free milli-Q water. The reconstituted vivo-morpholinos were kept in solution at room temperature. To test the vivo-morpholinos, A549 cells were seeded in 6 well plates at a density of 0.5×10\hat{ }6 cells per well in a final volume of 3 mL per well. The day after cell seeding, the vivo-morpholinos were diluted down to a final concentration of 4 μM and a final volume of 2 mL using 1× Opti-MEM™ I (Thermo Fisher Scientific, Waltham, MA), the plating media was discarded using vacuum suction, and the diluted vivo-morpholinos were immediately added to the cells (2 mL per well). The cells were maintained in the presence of the vivo-morpholinos at 37° C. and 5% CO 2 for another 24 hours and subsequently the cells were collected and processed for RNA isolation. Cell collection was achieved by removing the media containing the vivo-morpholinos and immediately adding 600 μL of Buffer RLT to the cells. Total RNA was purified using the QIAGEN RNeasy Mini Kit, using the QIAshredder homogenizer before loading the cell lysate onto the RNeasy spin columns, according to the instructions provided by the manufacturer (QIAGEN Inc., Germantown, MD). RNA quantification was performed using a NanoDrop™ One s Microvolume UV-Vis Spectrophotometer (Thermo Fisher Scientific). Upon quantification, the purified RNA was divided into 9 μL aliquots and stored at −80° C. until used for qRT-PCR.

qRT-PCR reactions were performed to assess the copy number estimates per 100 ng of total RNA (CNE_{100 ng}) for each one of the mature mRNA transcripts derived from the SUMO2 gene, that is, S2V1 and S2V2. To this end, embodiments of this disclosure can use the iTaq Universal SYBR® Green One-Step Kit (Bio-Rad Laboratories, Hercules, CA), following the manufacturer's protocol.

CNE_{100 ng} were calculated using the Cq values obtained in the qRT-PCR reactions indicated above and calibration curves generated using purified RNA produced in vitro corresponding to each of the segments targeted by the primer sets indicated above.

Primer Sets Used to Quantify the SUMO2 Transcripts:

The primer sets used to quantify each of the SUMO2 mature mRNA transcripts are indicated below.

S2V1 transcript:
Forward primer: S2V1V2.FW
SEQ ID NO: 7
5′ ACGAAAAGCCCAAGGAAGGAGTCAAG 3′
Reverse primer: S2V1.RV
SEQ ID NO: 8
5′ TGTATCTTCATCCTCCATTTCCAACTGTGC 3′
S2V2 transcript:
Forward primer: S2V1V2.FW
SEQ ID NO: 7
5′ ACGAAAAGCCCAAGGAAGGAGTCAAG 3′
Reverse primer: S2V2.RV
SEQ ID NO: 9
5′ TGTATCTTCATCCTCCATTTCCAACTGTCG 3′

The specificity of the qRT-PCR reactions performed was confirmed by agarose gel electrophoresis and DNA sequencing analyses of the PCR products generated. Notice that the forward primer used in both reactions was the same and that the reverse primers only differ from each other in the last two nucleotides at the 3′ end of their sequence.

Cycling Conditions:
i) Ramp 4° C./sec, 50° C., hold 600 secs.
ii) Ramp 5° C./sec, 95° C., hold 180 secs.
iii) Ramp 5° C./sec, 95° C., hold 10 secs.
iv) Ramp 4° C./sec, 60° C., hold 30 secs, acquire data, go back to step (iii) 39 additional times.
v) Ramp 4° C./sec, 60° C., hold 60 secs; ramp 0.05° C./sec, 95° C. hold 1 sec (high resolution melting analysis).

Data Obtained:

The experiments described above were performed four different times. The first experiment was done without using duplicates, so only single measurements were performed and therefore the data was not included in the data summary provided below. In the three subsequent independent experiments, all samples were analyzed in triplicate and the averages were calculated. All data obtained were tabulated in an Excel document; the Cq values entered were used to calculate the CNE_{100 ng} using calibration curves generated using purified RNA produced in vitro as indicated above. The Excel document produced is available upon request. The data obtained is summarized in FIG. 3 below.

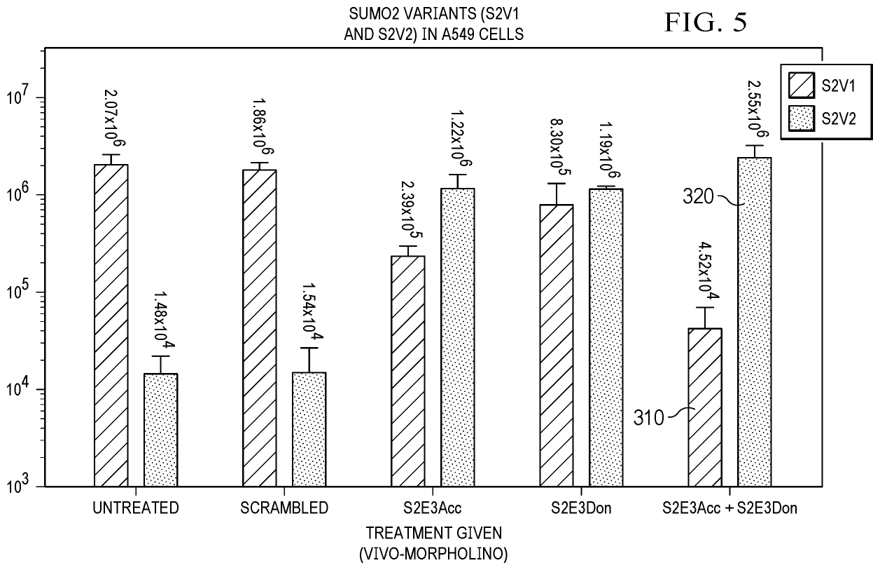

Referring to FIG. 5, quantitative analysis of the SUMO2 transcript variants present in A549 cells under normal conditions as well as upon a 24 hour treatment with the vivo-morpholinos S2E3Acc and S2E3Don is presented. The unexpected, commercially advantageous results can be comprehended by bars representing the average CNE_{100 ng} calculated for the S2V1 and S2V2 mRNAs, with the actual average values presented above the bars. The error bars indicate standard deviations. The treatments given (vivo-morpholino added to the cells) are indicated in the X axis. The data provided was obtained in three independent experiments, each performed in triplicate.

Under normal conditions, the S2V1 transcript is present at a CNE_{100 ng} of ˜2×10\hat{ }6, whereas S2V2 is present at a CNE_{100 ng} of ˜1.5×10\hat{ }4 (FIG. 5, Untreated sample). This represents a difference of a bit more than 100-fold in the concentration of these two mature mRNAs in the cell. S2V1 codes for the prototypical SUMO2, which is the predominant SUMO paralog present in A549 cells, as well as in all cell lines analyzed up to date. The S2V2 mRNA variant is produced by an alternative splicing event that results in the skipping of exon 3; therefore, S2V2 codes for SUMO2α, an isoform of SUMO2 that lacks 24 amino acid residues, from Q51 to A72 in SUMO2. Our experiments demonstrated that SUMO2α is non-conjugatable.

Treatment of A549 cells with a control vivo-morpholino containing a scrambled sequence had no effect on the proportion of S2V1:S2V2 present in the cell (FIG. 5, Scrambled sample). In contrast, the two vivo-morpholinos targeting S2 triggered significative changes in the proportions of S2V1:S2V2, with S2E3Acc triggering a larger decrease in the CNE_{100 ng} of S2V1 (about a 10-fold decrease) (FIG. 5, S2E3Acc and S2E3Don samples). Importantly, simultaneous treatment with the two vivo-morpholinos, S2E3Acc and S2E3Don, triggered the largest decrease 310 in S2V1 and the largest increase 320 in S2V2: The CNE_{100 ng} for S2V1 was decreased by almost 100-fold, whereas the CNE_{100 ng} for S2V2 was increased by almost 200-fold (evidence of results FIG. 5, S2E3Acc+S2E3Don sample). These changes indicate a reversal of the normal proportion of S2V1:S2V2 present in the cell, in which the new values represent an inversion of the normal values, S2V2 becoming 100-fold more abundant than S2V1. Thus, under these conditions SUMO2α is likely to be produced at substantially higher amounts than under normal conditions. Considering that SUMO2α is not conjugatable, and that the synthesis of prototypical SUMO2 is likely to be dramatically decreased by this change, treatment with S2E3Acc and S2E3Don is likely to result in an almost complete inactivation of the cellular SUMOylation system.

### Auxiliary Descriptions

A method, comprising quantifying SUMO2 transcripts comprising: maintaining mammalian cells in a presence of at least 2 vivo-morpholinos; and performing qRT-PCR reactions on RNA isolated from mammalian cells to assess copy number estimates per unit mass of total RNA for at least one mature mRNA transcript variant derived from SUMO2 selected from the group consisting of S2V1 or S2V2. The method wherein the at least one mature mRNA transcript variant derived from SUMO2 comprises S2V1 and S2V2. The method wherein the at least 2 vivo-morpholinos comprise: an S2E3Acc comprising a sequence of SEQ ID NO: 1; and an S2E3Don comprising a sequence of SEQ ID NO: 2. Further comprising: providing, before performing, a forward primer S2V1V2.FW comprising a sequence of SEQ ID NO: 3; and providing, before performing, a reverse primer S2V1.RV comprising a sequence of SEQ ID NO: 4. Further comprising: providing, before performing, a forward primer S2V1V2.FW comprising a sequence of SEQ ID NO: 7; and providing, before performing, a reverse primer S2V2.RV comprising a sequence of SEQ ID NO: 9.

### Definitions

The term uniformly is intended to mean unvarying or deviating very little from a given and/or expected value (e.g., within 10% of). The term substantially is intended to mean largely but not necessarily wholly that which is specified. The term approximately is intended to mean at least close to a given value (e.g., within 10% of). The term generally is intended to mean at least approaching a given state. The term coupled is intended to mean connected, although not necessarily directly, and not necessarily mechanically.

The terms first or one, and the phrases at least a first or at least one, are intended to mean the singular or the plural unless it is clear from the intrinsic text of this document that it is meant otherwise. The terms second or another, and the phrases at least a second or at least another, are intended to mean the singular or the plural unless it is clear from the intrinsic text of this document that it is meant otherwise. Unless expressly stated to the contrary in the intrinsic text of this document, the term or is intended to mean an inclusive or and not an exclusive or. Specifically, a condition A or B is satisfied by any one of the following: A is true (or present) and B is false (or not present), A is false (or not present) and B is true (or present), and both A and B are true (or present). The terms a and/or an are employed for grammatical style and merely for convenience.

The term plurality is intended to mean two or more than two. The term any is intended to mean all applicable members of a set or at least a subset of all applicable members of the set. The phrase any integer derivable therein is intended to mean an integer between the corresponding numbers recited in the specification. The phrase any range derivable therein is intended to mean any range within such corresponding numbers. Unless otherwise defined, all technical and scientific terms used herein have the same meaning as commonly understood by one of ordinary skill in the art to which this present disclosure belongs. In case of conflict, the present specification, including definitions, will control.

The described embodiments and examples are illustrative only and not intended to be limiting. Although embodiments of the present disclosure can be implemented separately, embodiments of the present disclosure may be integrated into the system(s) with which they are associated. All the embodiments of the present disclosure disclosed herein can be made and used without undue experimentation in light of the disclosure. Embodiments of the present disclosure are not limited by theoretical statements (if any) recited herein. The individual steps of embodiments of the present disclosure need not be performed in the disclosed manner, or combined in the disclosed sequences, but may be performed in any and all manner and/or combined in any and all sequences. Homologous replacements may be substituted for the substances described herein. Agents which are both chemically and physiologically related may be substituted for the agents described herein where the same or similar results would be achieved.

Various substitutions, modifications, additions and/or rearrangements of the features of embodiments of the present disclosure may be made without deviating from the scope of the underlying inventive concept. All the disclosed elements and features of each disclosed embodiment can be combined with, or substituted for, the disclosed elements and features of every other disclosed embodiment except where such elements or features are mutually exclusive. The scope of the underlying inventive concept as defined by the appended claims and their equivalents cover all such substitutions, modifications, additions and/or rearrangements.

The appended claims are not to be interpreted as including means-plus-function limitations, unless such a limitation is explicitly recited in a given claim using the phrase(s) “means for” or “mechanism for” or “step for”. Sub-generic embodiments of this disclosure are delineated by the appended independent claims and their equivalents. Specific embodiments of this disclosure are differentiated by the appended dependent claims and their equivalents.

What is claimed is:

1. A method of preventing, treating or delaying progression of cancer, comprising administering a therapeutically effective amount of a compound comprising at least 2 morpholinos to a patient in need thereof; wherein administering at least 2 vivo-morpholinos comprises administering:
an S1E2Acc comprising a sequence of SEQ ID NO: 1; or
an S1E2Don comprising a sequence of SEQ ID NO: 2.

2. The method of claim 1, wherein administering the compound comprises administering at least 2 vivo-morpholinos.

3. The method of claim 2, wherein each of the at least 2 vivo-morpholinos comprises an octa-guanidine dendrimer.

4. The method of claim 1, further comprising both reducing S2V1 in the patient and increasing S2V2 in the patient.

5. The method of claim 1, further comprising reducing S2V1 in the patient.

6. The method of claim 1, further comprising increasing S2V2 in the patient.

7. The method of claim 1, wherein administering at least 2 morpholinos comprises administering:
an S1E2Acc comprising a sequence of SEQ ID NO: 1; and
an S1E2Don comprising a sequence of SEQ ID NO: 2.

8. The method of claim 7, further comprising both reducing S2V1 in the patient and increasing S2V2 in the patient.

9. The method of claim 1, further comprising inhibiting establishment of chemoreistance.

10. A method of preventing, treating or delaying progression of cancer, comprising administering a therapeutically effective amount of a compound comprising at least 2 morpholinos to a patient in need thereof; wherein administering at least 2 vivo-morpholinos comprises administering:
an S2E3Acc comprising a sequence of SEQ ID NO: 3; or
an S2E3Don comprising a sequence of SEQ ID NO: 4.

11. The method of claim 10, wherein administering the compound comprises administering at least 2 vivo-morpholinos.

12. The method of claim 11, wherein each of the at least 2 vivo-morpholinos comprises an octa-guanidine dendrimer.

13. The method of claim 10, further comprising both reducing S2V1 in the patient and increasing S2V2 in the patient.

14. The method of claim 10, further comprising reducing S2V1 in the patient.

15. The method of claim 10, further comprising increasing S2V2 in the patient.

16. The method of claim 10, wherein administering at least 2 morpholinos comprises administering:
an S2E3Acc comprising a sequence of SEQ ID NO: 3; and
an S2E3Don comprising a sequence of SEQ ID NO: 4.

17. The method of claim 16, further comprising both reducing S2V1 in the patient and increasing S2V2 in the patient.

18. The method of claim 10, further comprising inhibiting establishment of chemoreistance.

19. A method of preventing, treating or delaying progression of cancer, comprising administering a therapeutically effective amount of a compound comprising at least 2 morpholinos to a patient in need thereof; wherein administering at least 2 vivo-morpholinos comprises administering:
an S3E3Acc comprising a sequence of SEQ ID NO: 5; or
an S3E3Don comprising a sequence of SEQ ID NO: 6.

20. The method of claim 19, wherein administering the compound comprises administering at least 2 vivo-morpholinos.

21. The method of claim 20, wherein each of the at least 2 vivo-morpholinos comprises an octa-guanidine dendrimer.

22. The method of claim 19, further comprising both reducing S2V1 in the patient and increasing S2V2 in the patient.

23. The method of claim 19, further comprising reducing S2V1 in the patient.

24. The method of claim 19, further comprising increasing S2V2 in the patient.

25. The method of claim 19, wherein administering at least 2 morpholinos comprises administering:
an S3E3Acc comprising a sequence of SEQ ID NO: 5; and
an S3E3Don comprising a sequence of SEQ ID NO: 6.

26. The method of claim 25, further comprising both reducing S2V1 in the patient and increasing S2V2 in the patient.

27. The method of claim 19, further comprising inhibiting establishment of chemoreistance.

28. A composition of matter, comprising a compound comprising at least 2 morpholinos comprising:
an S1E2Acc comprising a sequence of SEQ ID NO: 1; or
an S1E2Don comprising a sequence of SEQ ID NO: 2.

29. The composition of matter of claim 28, wherein the at least 2 morpholinos comprises at least 2 vivo-morpholinos.

30. The composition of matter of claim 29, wherein each of the at least 2 vivo-morpholinos comprises an octa-guanidine dendrimer.

31. The composition of matter of claim 1, wherein the at least 2 morpholinos comprise:
an S1E2Acc comprising a sequence of SEQ ID NO: 1; and
an S1E2Don comprising a sequence of SEQ ID NO: 2.

32. A composition of matter, comprising a compound comprising at least 2 morpholinos comprising:
an S2E3Acc comprising a sequence of SEQ ID NO: 3; or
an S2E3Don comprising a sequence of SEQ ID NO: 4.

33. The composition of matter of claim 32, wherein the at least 2 morpholinos comprises at least 2 vivo-morpholinos.

34. The composition of matter of claim 33, wherein each of the at least 2 vivo-morpholinos comprises an octa-guanidine dendrimer.

35. The composition of matter of claim 32, wherein the at least 2 morpholinos comprise:
an S2E3Acc comprising a sequence of SEQ ID NO: 3; and
an S2E3Don comprising a sequence of SEQ ID NO: 4.

36. A composition of matter, comprising a compound comprising at least 2 morpholinos comprising:
an S2E3Acc comprising a sequence of SEQ ID NO: 5; or
an S3E3Don comprising a sequence of SEQ ID NO: 6.

37. The composition of matter of claim 36, wherein the at least 2 morpholinos comprises at least 2 vivo-morpholinos.

38. The composition of matter of claim 37, wherein each of the at least 2 vivo-morpholinos comprises an octa-guanidine dendrimer.

39. The composition of matter of claim 36 wherein the at least 2 morpholinos comprise:
an S2E3Acc comprising a sequence of SEQ ID NO: 5; and
an S3E3Don comprising a sequence of SEQ ID NO: 6.


SEQUENCE LISTING (9 sequences)

SEQ ID NO: 1 - RNA, 25 nt, synthetic construct
  source 1..25: mol_type = other RNA
agttgaaggttttgcctcctgaaag

SEQ ID NO: 2 - RNA, 25 nt, synthetic construct
  source 1..25: mol_type = other RNA
caatgaaactcacctgtccaatgac

SEQ ID NO: 3 - RNA, 25 nt, synthetic construct
  source 1..25: mol_type = other RNA
cctcattgacaatccctgaacgaga

SEQ ID NO: 4 - RNA, 25 nt, synthetic construct
  source 1..25: mol_type = other RNA
gagatttttttacctgtgcaggtgt

SEQ ID NO: 5 - RNA, 25 nt, synthetic construct
  source 1..25: mol_type = other RNA
cctcattgacaagccctggaaagga

SEQ ID NO: 6 - RNA, 25 nt, synthetic construct
  source 1..25: mol_type = other RNA
tccgtacctgtgctggagtgtcagt

SEQ ID NO: 7 - RNA, 26 nt, synthetic construct
  source 1..26: mol_type = other RNA
acgaaaagcccaaggaaggagtcaag

SEQ ID NO: 8 - RNA, 30 nt, synthetic construct
  source 1..30: mol_type = other RNA
tgtatcttcatcctccatttccaactgtgc

SEQ ID NO: 9 - RNA, 30 nt, synthetic construct
  source 1..30: mol_type = other RNA
tgtatcttcatcctccatttccaactgtcg